(19) United States
(12) Patent Application Publication
(10) Pub. No.: US 2014/0065706 A1
(43) Pub. Date: Mar. 6, 2014

(54) MATERIALS AND METHODS FOR MOBILIZING CELLS INCLUDING CD34+, HEMATOPOIETIC COLONY FORMING, AND ENDOTHELIAL COLONY FORMING CELLS

(71) Applicants: Louis M. Pelus, Indianapolis, IN (US); Jonathan Hoggatt, Cambridge, MA (US)

(72) Inventors: Louis M. Pelus, Indianapolis, IN (US); Jonathan Hoggatt, Cambridge, MA (US)

(73) Assignee: Indiana University Research and Technology Corp., Indianapolis, IN (US)

(21) Appl. No.: 13/883,462

(22) PCT Filed: Nov. 7, 2011
(86) PCT No.: PCT/US2011/059622

§ 371 Date: Nov. 19, 2013

Related U.S. Application Data
(60) Provisional application No. 61/410,816, filed on Nov. 5, 2010.

(51) Int. Cl. C12N 5/078

(52) U.S. Cl. CPC C12N 5/0634
USPC 435/325

(57) ABSTRACT

Disclosed herein are materials and methods such as the use of none steroidal anti-inflammatory drugs to increase the number of CD34+ cell in human patients. The level of ECFC measured in volunteers before and after treatment with meloxicam. Only 4/7 volunteers had detectable levels of ECFC in a 40 mL sample of peripheral of blood (PB) before treatment, while 7/7 had detectable levels of ECFC in a similar sample collected post-meloxicam treatment. Administering a course of treatment with NSAID also increased the number of endothelial colony forming cells (ECFC) in the volunteers PB and it reduced culture time to first ECFC detection. These data demonstrate that NSAID administration mobilizes both hematopoietic and endothelial progenitors to PB. This provides a safe and effective strategy that can be added to existing mobilization regimens and is especially useful when combined with a course of treatment that includes the use of G-CSF.

### PRIORITY CLAIM

The application claims the benefit of U.S. Provisional Patent Application No. 61/410,816, filed on Nov. 5, 2010, which is hereby incorporated by reference in its entirety.

### STATEMENT OF GOVERNMENTAL RIGHTS

This invention was made with government support under grant number HL 069669 and under grant number HL096305 awarded by the National Institutes of Health. The United States Government has certain rights in the invention.

### FIELD OF THE INVENTION

This invention relates to using compounds such as non-steroidal anti-inflammatory drugs to aid in the mobilization of cells such as CD34+ cells, hematopoietic colony forming cells and endothelial colony forming cells in a patient or donor.

### BACKGROUND

Bone marrow progenitor cells have been implicated in neoangiogenesis. Circulating endothelial progenitor cells (EPC) have been found at low levels in mouse marrow and peripheral blood (PB) (Orlic et al., 2001a; Orlic et al., 2001b; Aicher et al., 2005) and in the CD34+ populations from adult PB and umbilical cord blood (UCB) (Asahara et al., 1997; Rafli and Lyden, 2003) and can be mobilized by ischemic injury, cytokines such as granulocyte macrophage colony stimulating factor (GM-CSF) and granulocyte colony forming factor (G-CSF), stromal cell derived factor 1 (SDF1) and vascular endothelial growth factor (VEGF) (Orlic et al., 2001b; Asahara et al., 1999; Takahashi et al., 1999; Aicher et al., 2005; Wolfram et al., 2007; Powell et al., 2005; Hattori et al., 2001). EPC mobilization represents a possible novel therapeutic option to enhance neovascularization. In animal models, mouse and human EPC have been shown to partially rescue cardiovascular dysfunction following ischemic hind limb or myocardial injury, with evidence for contribution to new vessel growth and re-endothelialization (Rafii and Lyden, 2003; Hu et al., 2006; Dimmeler et al., 2005; Orlic et al., 2001b). Clinically, intracoronary EPC administration has resulted in enhanced neovascularization with beneficial post-infarct remodeling (Strauer et al., 2002; Assmus et al., 2002). Given the great potential for the use of these cells in treating pathologies in mammals there remains a need for additional materials and methods that can help to increase the levels of these cells in peripheral blood; some aspects of the present invention seek to address these needs.

### SUMMARY

Some aspects of the invention related to methods of increasing the number of CD34+ and endothelial colony forming cells (ECFC) in the peripheral blood (PB) of a patient or donor with a therapeutically effective course of at least one non-steroidal anti-inflammatory drug (NSAID) for at least 4 consecutive days, which decreases the activity of PGE_{2.}

In some embodiments, the non-steroidal anti-inflammatory compound is selected from the group consisting of: aspirin, celecoxib, rofecoxib, etoricoxib, valdecoxib, ibuprofen, naproxen, diclofenac, etodolac, ketorolac, indomethacin, meloxicam and licofelone and pharmaceutically acceptable salts thereof. In some embodiments, the NSAID is indomethacin or a pharmaceutically acceptable salt thereof. And, in still other embodiments, the NSAID is meloxicam or a pharmaceutically acceptable salt thereof.

In some embodiments, a therapeutically effective course of NSAID is given in combination with granulocyte-colony stimulating factor or its structurally related analogs. Recombinant methionyl G-CSF, referred to as filgrastim is marketed by Amgen under the trade name Neupogen®.

Some embodiments of the invention include methods of mobilizing CD34+ cells, hematopoietic cells and endothelial progenitor cells or endothelial colony forming cells, comprising the steps of: treating a patient or a donor with an effective amount of at least one compound for at least 4 days wherein the compound that reduces the activity of at least one prostaglandin for at least 4 consecutive days; and collecting peripheral blood from the treated patient or donor and recovering a population of cells from the peripheral blood, wherein the population includes higher levels of, CD34+ cells, hematopoietic cells and endothelial progenitor cells or endothelial colony forming cells.

In some embodiments the compound that reduces the activity of at least one prostaglandin is a NSAID. In some embodiments the non-steroidal anti-inflammatory drug acts on at least one enzyme selected from the group consisting of: cyclooxygenase-1 and cyclooxygenase-2. In some embodiments the non-steroidal anti-inflammatory drug used to practice the invention includes at least one compound selected from the group consisting of: aspirin, celecoxib, rofecoxib, etoricoxib, valdecoxib, ibuprofen, naproxen, diclofenac, etodolac, ketorolac, indomethacin, meloxicam and licofelone.

In some embodiments of the invention the non-steroidal anti-inflammatory compound used to practice the invention is indomethacin or a pharmaceutically acceptable salt thereof. In some embodiment the non-steroidal anti-inflammatory compound is meloxicam or a pharmaceutically acceptable salt thereof. In some embodiments of the invention the therapeutically effective dose of the non-steroidal anti-inflammatory compound is between about 0.5 to about 50 mg per kg⁻¹. In some embodiment the therapeutically effective dose of meloxicam is between about 0.1 to about 6.0 mg per kg⁻¹.

Some embodiments of the invention include dosing the patient or donor with a therapeutically effective amount of G-CSF, wherein the G-CSF and the compound that reduces the activity of at least one prostaglandin such as an NSAID are administered to the patient or donor at the same time within the same therapeutic window. In some embodiments therapeutically effective amount of G-CSF is on the order of between about 100 μg per kg⁻¹ to about 0.1 μg per kg⁻¹ per day. And in still other embodiments of the invention the therapeutically effective amount of G-CSF is on the order of between about 50 μg per kg⁻¹ to about 0.5 μg per kg⁻¹ per day or between about 20 μg per kg⁻¹ to about 1.0 μg per kg⁻¹ per day.

In some embodiments of the invention the compound that alters the activity of PGE₂ and that is administered to the patient or donor is an antagonist of at least one PGE₂ receptor.

In some embodiments of the invention the antagonist of at least one PGE₂ receptor is at least one compound selected from the group consisting of: N-[[4′-[[3-butyl-1,5-dihydro-5-oxo-1-[2-(trifluoromethyl)phenyl]-4H-1,2,4-triazol-4-yl]methyl] [1,1′-biphenyl]-2-yl]sulfonyl]-3 -methyl-2-thiophenecarboxamide and 4-(4,9-diethoxy-1,3-dihydro-1-oxo-2H-benz[f]isoindol-2-yl)-N-(phenylsulfonyl)-benzeneacetamide.

### BRIEF DESCRIPTION OF THE FIGURES

FIG. 1A. Plot of CD34+ cells per mL of blood recovered from Baboons before and after 5 days of treatment with meloxicam.

FIG. 1B. Plot of CFU-GM cells per mL of blood recovered from Baboons before and after 5 days of treatment with meloxicam.

FIG. 2A. Plot of CD34+ cells per mL of blood recovered from Baboons before and after 5 days of treatment with meloxicam and G-CSF.

FIG. 2B. Plot of CFU-GM cells per mL of blood recovered from Baboons before and after 5 days of treatment with meloxicam and G-CSF.

FIG. 3A. Plot of CD34+ cells per mL of blood, recovered from human volunteers pre and post a 5 day course of treatment with meloxicam.

FIG. 3B. Plot of CFU-GM cells per mL of blood, recovered from human volunteers pre and post a 5 day course of treatment with meloxicam.

FIG. 3C. Plot of BFU-E per mL of blood, recovered from human volunteers pre and post a 5 day course of treatment with meloxicam.

FIG. 3D. Plot of CFU-GEMM per mL of blood, recovered from human volunteers pre and post a 5 day course of treatment with meloxicam.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

As used herein, unless explicitly stated otherwise or clearly implied otherwise the term ‘about’ refers to a range of values plus or minus 10 percent, e.g. about 1.0 encompasses values from 0.9 to 1.1

As used herein, unless explicitly stated otherwise or clearly implied otherwise the terms ‘therapeutically effective dose,’ ‘therapeutically effective amounts,’ and the like, refers to a portion of a compound that has a net positive effect on the health and well being of a human or other animal. Therapeutic effects may include an improvement in longevity, quality of life and the like these effects may also include a reduced susceptibility to developing disease or deteriorating health or well being. The effects may be immediately realized after a single dose and/or treatment or they may be cumulatively realized after a series of doses and/or treatments.

Determining the optimal dosing level and/or course of treatment for a given human or animal patient is dependent on a number of well known factors including the age, size, species, and health of the human or animal patient being treated. Given, data for a group of mammalian patients it is well within the skills of the clinician of ordinary skill in a given medical or veterinary specialty to determine an advantageous dosing level or course, barring any unexpected results, without having to engage in undue experimentation.

Dosing units as used herein are generally given in units of mass of the active ingredient per kilogram of the patient's or donor's body mass; e.g. mg of NSAID per kg (mg kg⁻¹) of the patient's body mass.

Pharmaceutically acceptable salts include salts of compounds of the invention that are safe and effective for use in mammals and that possess a desired therapeutic activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention may form pharmaceutically acceptable salts with various amino acids. Suitable base salts include, but are not limited to, aluminium, calcium, lithium, magnesium, potassium, sodium, zinc, and diethanolamine salts. For additional information on some pharmaceutically acceptable salts that can be used to practice the invention please reviews such as Berge, et al., 66 J. PHARM. SCI. 1-19 (1977), Haynes, et al, J. Pharma. Sci., Vol. 94, No. 10, October 2005, pgs. 2111-2120 and the like.

Some aspects of the invention include using compounds that can reduce the effect of PGE₂ on certain cell types. These compounds, include, but are not limited to, wherein the compound that reduces PGE₂ activity is a non-steroidal anti-inflammatory compound. This class includes compounds that act on at least one enzyme selected from the group consisting of: cyclooxygenase-1 and cyclooxygenase-2. In some embodiments, the steroidal anti-inflammatory compound acts primarily on cyclooxygenase-2. In some embodiments, the non-steroidal anti-inflammatory compound is selected from the group consisting of: aspirin, celecoxib, rofecoxib, etoricoxib, valdecoxib, ibuprofen, naproxen, diclofenac, etodolac, ketorolac, indomethacin, meloxicam and licofelone. In some embodiment, the non-steroidal anti-inflammatory compound is indomethacin. While in others, it may be or may include meloxicam.

In some embodiments, the compound that alters the active of PGE₂ is an antagonist of at least one PGE₂ receptor. In some embodiments, these antagonist of at least one PGE₂ receptor may be selected from the groups consisting of: N-[[4′-[[3-butyl-1,5-dihydro-5-oxo-1-[2-(trifluoromethyl)phenyl]-4H-1,2,4-triazol-4-yl]methyl][1,1′-biphenyl]-2-yl]sulfonyl]-3-methyl-2-thiophenecarboxamide and 4-(4,9-diethoxy-1,3-dihydro-1-oxo-2H-benz[f]isoindol-2-yl)-N-(phenylsulfonyl)-benzeneacetamide.

For additional information see, for example, International Publication Number WO 2010/054271 A1 based on International Application No. PCT/US2009/063654, having an International Filing Date of Nov. 6, 2009 entitled “Materials and Methods to Enhance Hematopoietic Stem Cells Engraftment Procedures” to Pelus, et al., which itself claims the benefit of U.S. provisional patent application No. 61/112,018 filed on Nov. 6, 2008 and also see, for example, International Publication No. WO 2011/060381 A1, based on International Application No. PCT/US2010/056744, having an International Filing Date of Nov. 15, 2010, entitled “Methods to Enhance Delivery and Engraftment of Stem Cells Including the Identification of Specific Prostagandin E₂ Receptors”, to Pelus, et al., which itself claims the benefit of U.S. provisional patent application No. 61/261,352 filed on Nov. 15, 2009 and U.S. provisional patent application No. 61/261,349 filed on Nov. 15, 2009, each of these International Patent Applications and U.S. provisional patent applications are incorporated herein by reference in its entirety as if each were incorporated individually in its entirety.

EPC mobilization represents a possible novel therapeutic option to enhance neovascularization. In animal models, mouse and human EPC have been shown to partially rescue cardiovascular dysfunction following ischemic hind limb or myocardial injury, with evidence for contribution to new vessel growth and re-endothelialization (Rafii and Lyden, 2003; Hu et al., 2006; Dimmeler et al., 2005; Orlic et al., 2001b). Clinically, intracoronary EPC administration has resulted in enhanced neovascularization with beneficial post-infarct remodeling (Strauer et al., 2002; Assmus et al., 2002). However, while some benefit has been observed, there is little evidence for EPC engraftment in newly formed blood vessels (Dimmeler et al., 2005; Hristov and Weber, 2006). Since most EPC populations are defined by biomarkers that are shared by hematopoietic cells and also contain CD14+ monocytic cells, it is likely that reported positive effects result from paracrine effects of hematopoietic cells (HSPC) driving the angiogenic process (Kopp et al., 2006; Willett et al., 2005). A critical issue currently restricting the field is a lack of unique biomarkers to define EPC (Yoder, 2009) and it is absolutely imperative to define EPC at a clonogenic level. In addition, the fact that potent mobilizers such as G-CSF also exert pro-inflammatory capacity that may enhance atherosclerosis in patients with coronary artery disease (Aicher et al., 2005), suggest the need for additional methods to enhance EPC frequency.

A functional approach has been used to define a rare population of circulating endothelial colony forming cells (ECFC) in adult human PB that appear to be true EPC (Yoder et al., 2007; Ingram et al., 2004). ECFC express markers of primary endothelium (CD31,105,144,146, VWF, KDR and UEA1) but lack most hematopoietic cell markers. However, they are contained within the CD34+ fraction of human PB and UCB. These cells have not been identified in normal mouse or rat PB. Endothelial colonies derived from PB mononuclear cells clonally propagate with varying proliferative capacity, re-plate into secondary and tertiary ECFC, and form capillary structures in vitro, and human blood vessels in vivo in immune deficient mice with incorporation into the murine vasculature (Yoder et al., 2007)

### Exemplary Experiments and Results

### Effects of NSAIDS on Various Cell Lines Determined in Baboons

Baboons received 0.1 mg/kg Meloxicam daily for 5 days either alone or in combination with 10 microgram/kg G-CSF (Neupogen®). Peripheral blood was collected in EDTA and analyzed for CD34+ cells by FACS and CFU-GM in agar culture.

For analysis of circulating ECFC, PB mononuclear cells were plated in media at 37° C., 5% CO₂, 5% O₂ in 6 well culture plates coated with type 1 collagen. Media was be replaced every other day for 7-14 days at which time the adherent colonies of endothelial cells (EC) are removed with Trypsin and replated in collagen coated culture wells at a single cell per well. After 14 days each well is visually examined to determine whether there are 2 or more cells per well and if so, the total number of EC counted.

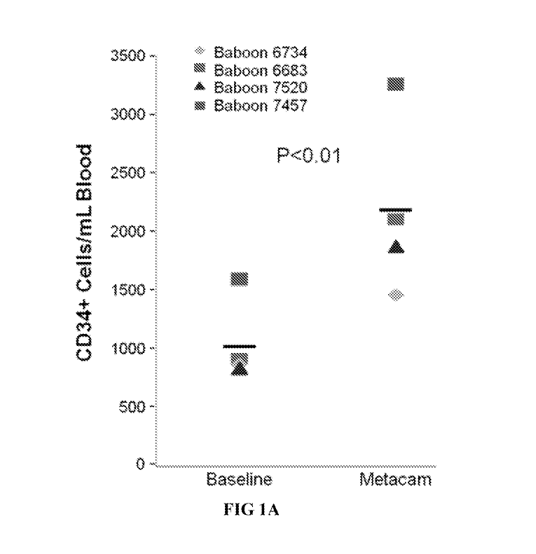

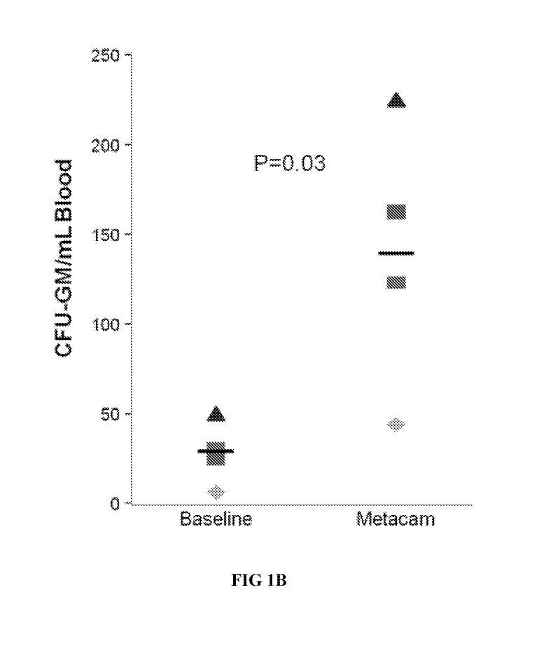

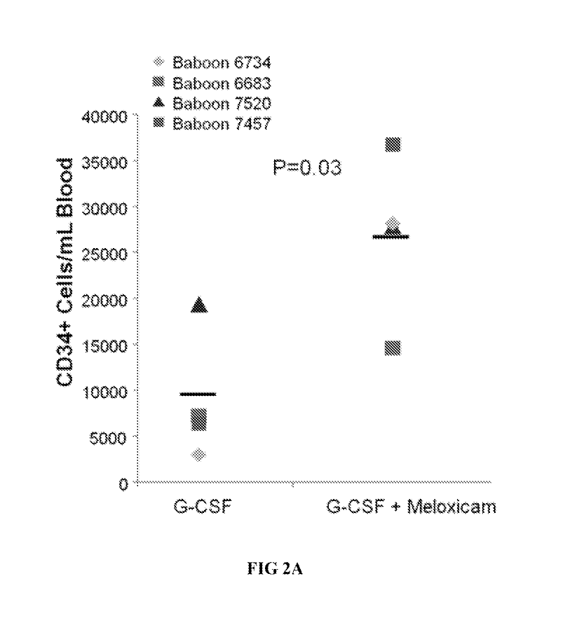

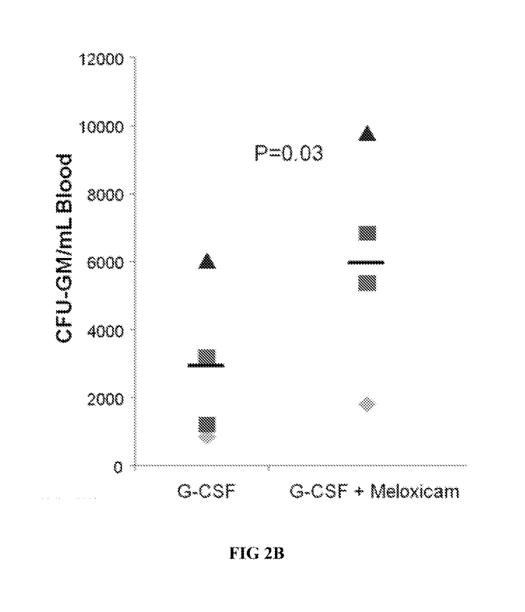

In mice, treatment with NSAIDS alone results in expansion and mobilization of HSPC and the combination of NSAID with G-CSF (Neupogen) synergistically enhances mobilization. Referring now to FIGS. 1A and 1B, in baboons, NSAIDS alone mobilize CD34+ cells and hematopoietic progenitor cells. Referring now to FIGS. 2A and 2B, NSAIDs were used in combination with G-CSF NSAIDs significantly enhance mobilization of CD34+ cells and hematopoietic progenitor cells.

### Effect of NSAID on Various Cell Lines in Humans

In an IRB approved study, a standard dose of 15 mg/day Meloxicam, a dose equivalent to the doses used in baboon and murine studies, was administered to normal human volunteers for 5 days. Peripheral blood was obtained pre and post treatment. CD34+ cells were enumerated by FACS and clonogenic progenitors quantitated in methylcellulose culture.

To determine if NSAIDs are also capable of HSPC mobilization in humans, peripheral blood (PB) of 7 healthy volunteers was analyzed for CD34+ cells by the ISHAGE protocol, and CFU-GM, BFU-E and CFU-GEMM in methylcellulose culture, before and after a 5 day regimen of the NSAID meloxicam. Meloxicam treatment resulted in significant mobilization of CD34+ cells, CFU-GM and CFU-GEMM (2.0±0.5, 3.8±0.9 and 3.4±0.7 fold, respectively; P<0.05) with no change in BFU-E, consistent with the erythropoietic stimulatory effect of PGE₂. In addition to hematopoiesis, marrow progenitors are also implicated in neoangiogenesis. Endothelial progenitors are found in mouse bone marrow and blood, and in the CD34+ adult and cord blood populations, and can be mobilized by ischemic injury and various cytokines and growth factors. Utilizing a functional approach to define a rare population of circulating endothelial colony forming cells (ECFC) as previously described (Yoder et al., 2007, Blood), we assessed the level of ECFC in volunteers before and after meloxicam administration. Only 4/7 volunteers had detectable ECFC in 40 mL of blood before treatment, while 7/7 had detectable ECFC post-meloxicam. In addition, NSAID treatment increased ECFC number and reduced culture time to first ECFC detection. These data demonstrate that NSAID administration alters the bone marrow niche and mobilizes both hematopoietic and endothelial progenitors to PB.

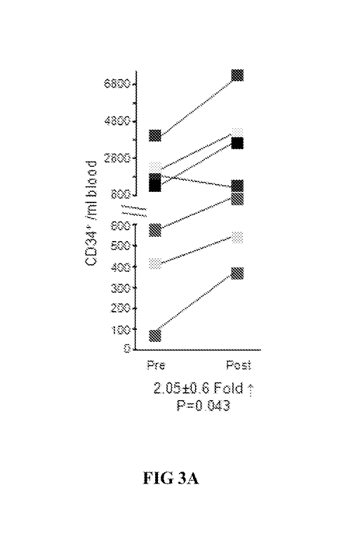

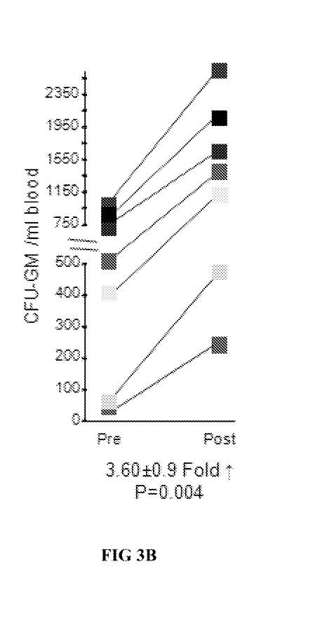

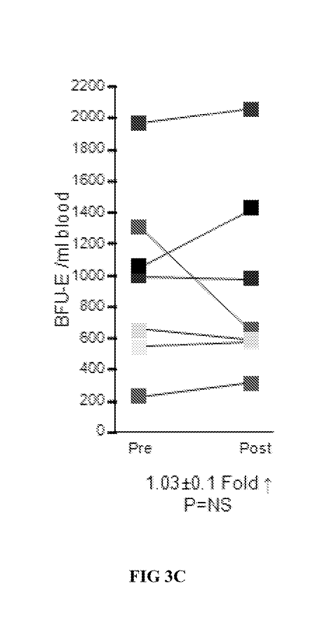

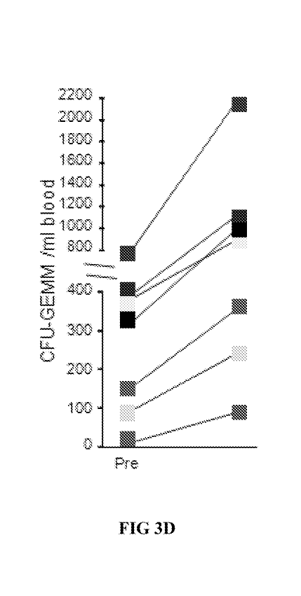

Referring now to FIGS. 3A, 3B, 3C and 3D, as with baboons, human volunteers mobilize CD34+ and CFU-GM and CFU-GEMM hematopoietic progenitors in response to treatment with Meloxicam alone. No change in erythroid progenitors (BFU-E) was observed, consistent with the erythropoietic stimulatory effect of PGE on hematopoietic progenitor cells.

The fact that we observe a significant increase in CD34+ cells in baboons and HSPC in mice treated with the NSAIDs suggests that NSAIDs may mobilize other progenitor cell types. Analysis of the rare ECFC that can contribute to neoangiogenesis and repair of ischemic injury showed that only 4 of 7 volunteers had detectable ECFC in 40 ml of PB before NSAID treatment while 7 of 7 volunteers had detectable ECFC post Meloxicam treatment. In addition, NSAID treatment increased ECFC by twofold and reduced culture time to first detection of ECFC by 4 days.

The fact that the mobilizing effect of NSAIDs alone on hematopoietic progenitors and CD34+ cells is equivalent in baboons and man and also observed for hematopoietic stem and progenitors cells in mice, and that like mice, enhanced mobilization of CD34+ and progenitor cells is seen with the combination of NSAID plus G-CSF in baboons, argues that NSAID plus G-CSF will result in enhanced mobilization in humans as well, since both mice and baboons are validated models for this effect in man. Since ECFC are contained within the CD34+ fraction, it is expected that combination of NSAID with G-CSF in man will result in increased CD34+ and ECFC.

This provides a safe and effective strategy that can be easily added to existing mobilization regimens to improve efficacy. Also disclosed is the ability of NSAIDs to increase peripheral ECFC; perhaps suggesting an exciting possible mechanism, in addition to inhibition of platelets, for the beneficial effects of NSAIDs like aspirin on the cardiovascular system. It is interesting to note that the highly COX2 selective NSAID valdecoxib was recently withdrawn from the market due to adverse cardiovascular events, and our studies demonstrate that valdecoxib is unable to mobilize progenitors from the bone marrow, while aspirin is highly effective, possibly explaining some of the cardiovascular differences between these two compounds.

### References

Aicher, A., Zeiher, A. M., and Dimmeler, S. (2005). Mobilizing endothelial progenitor cells. Hypertension 45, 321-325.
Asahara, T., Murohara, T., Sullivan, A., Silver, M., van der Zee, R., Li, T., Witzenbichler, B., Schatteman, G., and Isner, J. M. (1997). Isolation of putative progenitor endothelial cells for angiogenesis. Science 275, 964-967.
Asahara, T., Takahashi, T., Masuda, H., Kalka, C., Chen, D., Iwaguro, H., Inai, Y., Silver, M., and Isner, J. M. (1999). VEGF contributes to postnatal neovascularization by mobilizing bone marrow-derived endothelial progenitor cells. EMBO J. 18, 3964-3972.
Assmus, B., Schachinger, V., Teupe, C., Britten, M., Lehmann, R., Dobert, N., Grunwald, F., Aicher, A., Urbich, C., Martin, H., Hoelzer, D., Dimmeler, S., and Zeiher, A. M. (2002). Transplantation of Progenitor Cells and Regeneration Enhancement in Acute Myocardial Infarction (TOPCARE-AMI). Circulation 106, 3009-3017.
Dimmeler, S., Zeiher, A. M., and Schneider, M. D. (2005). Unchain my heart: the scientific foundations of cardiac repair. J. Clin. Invest 115, 572-583.
Hattori, K., Dias, S., Heissig, B., Hackett, N. R., Lyden, D., Tateno, M., Hicklin, D. J., Zhu, Z., Witte, L., Crystal, R. G., Moore, M. A., and Rafii, S. (2001). Vascular endothelial growth factor and angiopoietin-1 stimulate postnatal hematopoiesis by recruitment of vasculogenic and hematopoietic stem cells. J. Exp. Med 193, 1005-1014.
Hristov, M. and Weber, C. (2006). The therapeutic potential of progenitor cells in ischemic heart disease—Past, present and future. Basic Res. Cardiol. 101, 1-7.
Hu, L., Cheng, L., Wang, J., Zhao, H., Duan, H., and Lu, G. (2006). Effects of human yolk sac endothelial cells on supporting expansion of hematopoietic stem/progenitor cells from cord blood. Cell Biol. Int. 30, 879-884.
Ingram, D. A., Mead, L. E., Tanaka, H., Meade, V., Fenoglio, A., Mortell, K., Pollok, K., Ferkowicz, M. J., Gilley, D., and Yoder, M. C. (2004). Identification of a novel hierarchy of endothelial progenitor cells using human peripheral and umbilical cord blood. Blood 104, 2752-2760.
Kopp, H. G., Ramos, C. A., and Rafii, S. (2006). Contribution of endothelial progenitors and proangiogenic hematopoietic cells to vascularization of tumor and ischemic tissue. Curr. Opin. Hematol. 13, 175-181.
Orlic, D., Kajstura, J., Chimenti, S., Jakoniuk, I., Anderson, S. M., Li, B., Pickel, J., McKay, R., Nadal-Ginard, B., Bodine, D. M., Leri, A., and Anversa, P. (2001a). Bone marrow cells regenerate infarcted myocardium. Nature 410, 701-705.
Orlic, D., Kaj stura, J., Chimenti, S., Limana, F., Jakoniuk, I., Quaini, F., Nadal-Ginard, B., Bodine, D. M., Leri, A., and Anversa, P. (2001b). Mobilized bone marrow cells repair the infarcted heart, improving function and survival. Proc. Natl. Acad. Sci. U.S.A 98, 10344-10349.
Powell, T. M., Paul, J. D., Hill, J. M., Thompson, M., Benjamin, M., Rodrigo, M., McCoy, J. P., Read, E. J., Khuu, H. M., Leitman, S. F., Finkel, T., and Cannon, R. O., III (2005). Granulocyte colony-stimulating factor mobilizes functional endothelial progenitor cells in patients with coronary artery disease. Arterioscler. Thromb. Vasc. Biol. 25, 296-301.
Rafii, S. and Lyden, D. (2003). Therapeutic stem and progenitor cell transplantation for organ vascularization and regeneration. Nat. Med 9, 702-712.
Strauer, B. E., Brehm, M., Zeus, T., Kostering, M., Hernandez, A., Sorg, R. V., Kogler, G., and Wernet, P. (2002). Repair of infarcted myocardium by autologous intracoronary mononuclear bone marrow cell transplantation in humans. Circulation 106, 1913-1918.
Takahashi, T., Kalka, C., Masuda, H., Chen, D., Silver, M., Kearney, M., Magner, M., Isner, J. M., and Asahara, T. (1999). Ischemia- and cytokine-induced mobilization of bone marrow-derived endothelial progenitor cells for neovascularization. Nat. Med 5, 434-438.
Willett, C. G., Czito, B. G., Bendell, J. C., and Ryan, D. P. (2005). Locally advanced pancreatic cancer. J. Clin. Oncol. 23, 4538-4544.
Wolfram, O., Jentsch-Ullrich, K., Wagner, A., Hammwohner, M., Steinke, R., Franke, A., Zupan, I., Klein, H. U., and Goette, A. (2007). G-CSF-induced mobilization of CD34(+) progenitor cells and proarrhythmic effects in patients with severe coronary artery disease. Pacing Clin. Electrophysiol. 30 Suppl 1, S166-S169.
Yoder, M. C. (2009). Defining human endothelial progenitor cells. J. Thromb. Haemost. 7 Suppl 1, 49-52.
Yoder, M. O., Mead, L. E., Prater, D., Krier, T. R., Mroueh, K. N., Li, F., Krasich, R., Temm, C. J., Prchal, J. T., and Ingram, D. A. (2007). Redefining endothelial progenitor cells via clonal analysis and hematopoietic stem/progenitor cell principals. Blood 109, 1801-1809.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1. A method of mobilizing cells, comprising the steps of:
treating a patient or a donor with an effective amount of at least one compound for at least 4 days, wherein the compound reduces the activity of at least one prostaglandin for at least 4 consecutive days;
collecting peripheral blood from the treated patient or the donor; and
recovering a population of cells from the peripheral blood, wherein the population includes higher levels of, CD34+ cells, hematopoietic cells and endothelial progenitor cells or endothelial colony forming cells.

2. The method according to claim 1, wherein the compound that reduces the activity of at least one prostaglandin is a NSAID.

3. The method according to claim 2 wherein the non-steroidal anti-inflammatory acts on at least one enzyme selected from the group consisting of: cyclooxygenase-1 and cyclooxygenase-2.

4. The method according to claim 2, wherein the non-steroidal anti-inflammatory compound is selected from the group consisting of: aspirin, celecoxib, rofecoxib, etoricoxib, valdecoxib, ibuprofen, naproxen, diclofenac, etodolac, ketorolac, indomethacin, meloxicam and licofelone.

5. The method according to claim 2, wherein the non-steroidal anti-inflammatory compound is indomethacin or a pharmaceutically acceptable salt thereof.

6. The method according to claim 2, wherein the non-steroidal anti-inflammatory compound is meloxicam or a pharmaceutically acceptable salt thereof.

7. The method according to claim 4, wherein the therapeutically effective dose of said non-steroidal anti-inflammatory compound is about 0.5-50 mg per Kg⁻¹.

8. The method according to claim 6, wherein the therapeutically effective dose of meloxicam is about 0.1-6.0 mg per kg⁻¹.

9. The method according to claim 1, further including the step of:
dosing the patient or donor with a therapeutically effective amount of G-CSF, wherein the G-CSF and the compound that reduces the activity of at least one prostaglandin are administered to the patient or donor at the same time.

10. The method according to claim 9, wherein the therapeutically effective amount of G-CSF is on the order of between about 100 μg per kg⁻¹ to about 0.1 μg per kg⁻¹ per day.

11. The method according to claim 9, wherein the therapeutically effective amount of G-CSF is on the order of between about 50 μg per kg⁻¹ to about 0.5 μg per kg⁻¹ per day.

12. The method according to claim 9, wherein the therapeutically effective amount of G-CSF is on the order of between about 20 μg per kg⁻¹ to about 1.0 μg per kg⁻¹ per day.

13. The method according to claim 1, wherein the compound that alters the activity of PGE₂ is an antagonist of at least one PGE₂ receptor.

14. The method according to claim 13, wherein the antagonist of at least one PGE₂ receptor is selected from the group consisting of: N-[[4′-[[3-butyl-1,5-dihydro-5-oxo-1-[2-(trifluoromethyl)phenyl]-4H-1,2,4-triazol-4-yl]methyl][1,1′-biphenyl]-2-yl]sulfonyl]-3-methyl-2-thiophenecarboxamide and 4-(4,9-diethoxy-1,3-dihydro-1-oxo-2H-benz[f]isoindol-2-yl)-N-(phenylsulfonyl)-benzeneacetamide.
